(19) United States
(12) Patent Application Publication
(10) Pub. No.: US 2026/0008761 A1
(43) Pub. Date: Jan. 8, 2026

(54) PROCESS FOR THE PREPARATION OF TAXANE DERIVATIVES

(71) Applicant: Indena S.P.A., Milano (IT)

(72) Inventors: Vincenzo Rositano, Milano (IT); Luca Senaldi, Milano (IT); Andrea Gambini, Milano (IT); Anna Bernardi, Milano (IT)

(73) Assignee: Indena S.P.A., Milano (IT)

(21) Appl. No.: 18/695,504

(22) PCT Filed: Sep. 29, 2022
(86) PCT No.: PCT/IB2022/059286

§ 371 Date: Mar. 26, 2024

(30) Foreign Application Priority Data
Sep. 30, 2021 (IT) 102021000025172

(51) Int. Cl. C07D 305/14; C07C 67/08; C07C 69/34

(52) U.S. Cl. CPC C07D 305/14; C07C 67/08; C07C 69/34

(57) ABSTRACT

Disclosed is a process for the preparation of taxanes esterified with octadecanedioic acid which comprises reacting a taxane with an octadecanedioic acid protected in one of the two carboxyl groups and protected with a group removable by catalysis with transition metals.

### FIELD OF INVENTION

The present invention relates to a process for the preparation of high-purity taxane derivatives.

### BACKGROUND TO THE INVENTION

Taxanes are a class of antitumoral agents, widely used in chemotherapy, which stabilise microtubule polymerisation at cell level, thereby inhibiting mitosis; said compounds can be obtained, for example, from the bark of Taxus brevifolia by extraction and purification, or by semi-synthetic processes starting with 10-deacetylbaccatin of Formula (1). The taxanes most widely used in clinical practice are the following:

paclitaxel (“PTX”) of Formula (2), used to treat cancer of the lung, ovary, breast and head-neck region and advanced forms of Kaposi's sarcoma; docetaxel of Formula (3), used for operable breast cancer, non-small-cell lung cancer, prostate cancer and gastric adenocarcinoma; and cabazitaxel of Formula (4), used to treat metastatic castration-resistant prostate cancer. Although said antitumoral agents are widely used, their use is accompanied by a number of side effects, which can be severe.

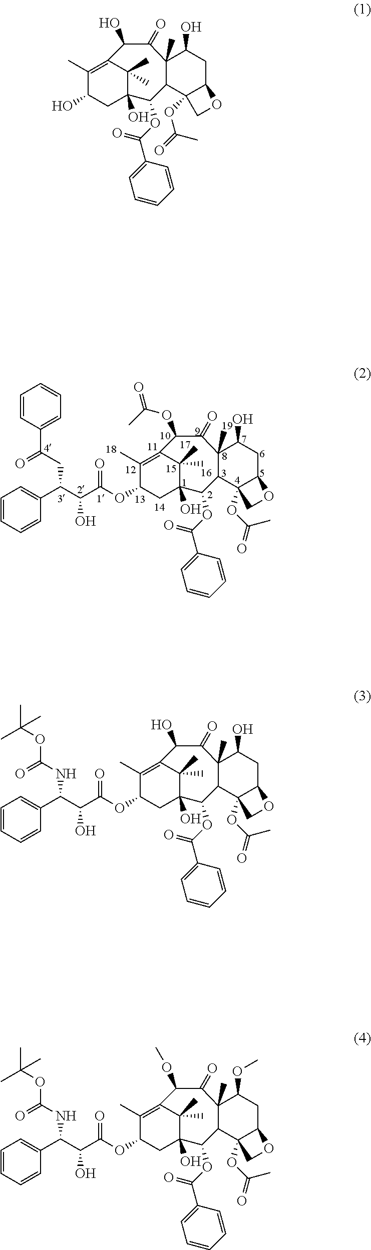

ODDA-paclitaxel (“ODDA-PTX”) of Formula (5) is a prodrug of paclitaxel (2); 5 said compound, following esterification with octadecanedioic acid of Formula (6) (“ODDA”), improves the non-covalent bond between paclitaxel (2) and human serum albumin, thereby reducing the side effects caused by damage to healthy tissues, and is preferably absorbed by some tumour cells that overexpress certain surface proteins, such as protein CD36, which promote the transport of fatty acids into the cell (WO 10 2021/007322).

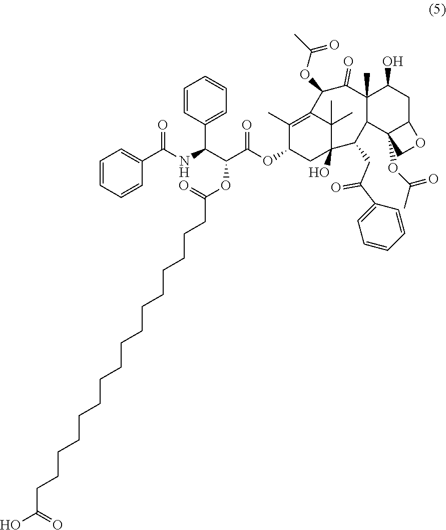

The properties of ODDA-paclitaxel (5) have been verified by in vivo tests, which demonstrate that it is more effective than the traditional formulations of paclitaxel (2) in the treatment of fibrosarcoma, pancreatic cancer and colon cancer (Callmann et al., J. Am. Chem. Soc. 2019, 141, 11765-11769).

Callmann et al. describe a process for the preparation of ODDA-paclitaxel (5) by esterification of octadecanedioic acid (6) and paclitaxel of Formula (2) in the presence of condensing agents such as ethyl-dimethylaminopropyl-carbodiimide (“EDC”) and dimethylaminopyridine (“DMAP”).

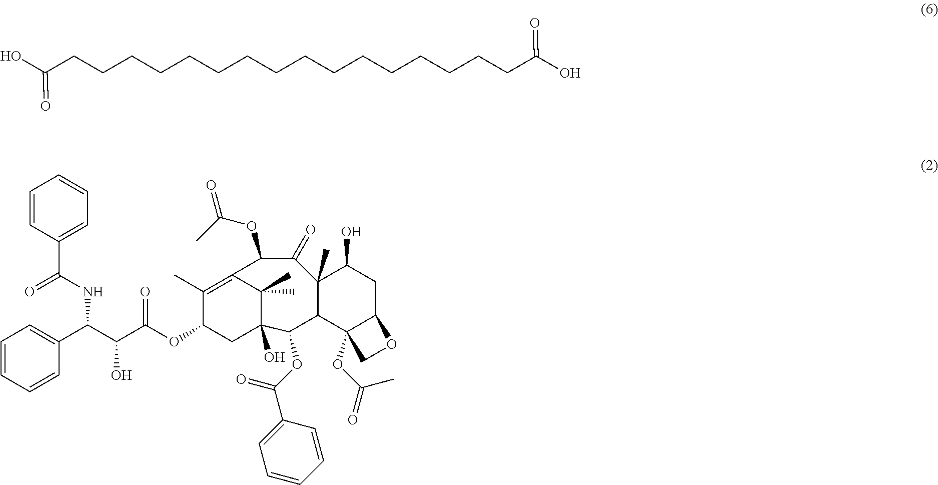

However, said synthesis approach provides ODDA-paclitaxel (5) with low yields, due to the formation of by-products such as the condensate paclitaxel-ODDA-paclitaxel of Formula (7) (“PTX-ODDA-PTX”), and ODDA-paclitaxel-ODDA (“ODDA-PTX-ODDA”) of Formula (8), wherein esterification of the paclitaxel hydroxyls (2) present at the 2′ and 7 positions takes place.

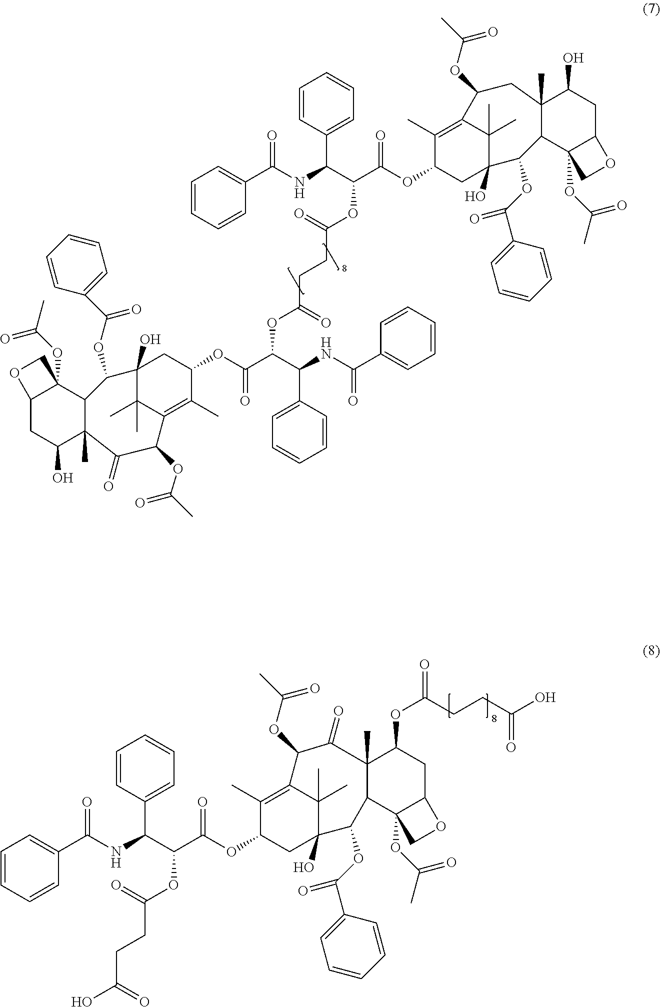

An alternative synthesis process, exemplified in WO 2017/053391 and WO 2021/007322, involves initial conversion of ODDA (6) to the mono-triisopropylsilylester of Formula (9),

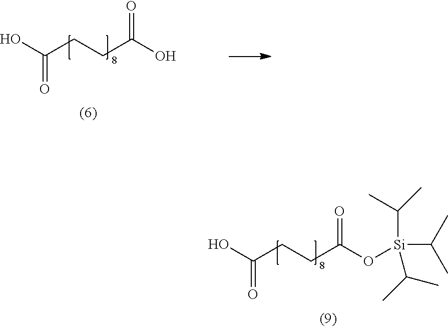

followed by esterification of paclitaxel (2) with the compound of Formula (9) to give the compound of Formula (10)

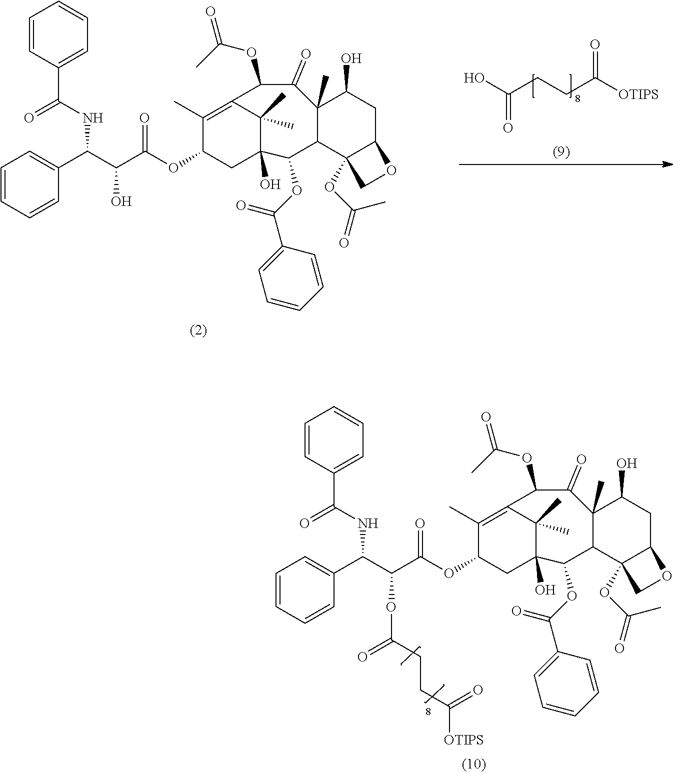

10 and finally, removal of the silicon protecting group to obtain ODDA-paclitaxel (5).

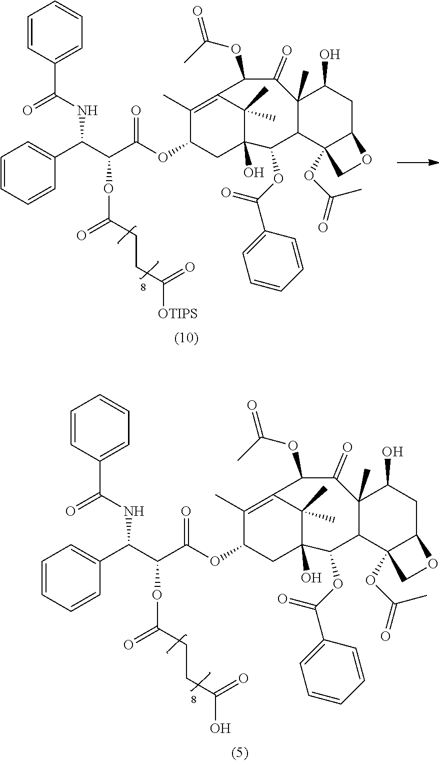

Said synthesis method prevents formation of the impurity PTX-ODDA-PTX (7) due to the use of compound (9), but not formation of ODDA-PTX-ODDA (8).

Moreover, further undesirable derivatives may form due to the presence in commercial ODDA of impurities such as diacids with chains having a larger or smaller number of carbon atoms. There is therefore still a need to develop a process that provides ODDA-PTX (5), and possibly other ODDA-taxanes, with high purity.

### DESCRIPTION OF THE INVENTION

The applicant has developed a novel process for the preparation of ODDA-taxanes of general Formula (11)

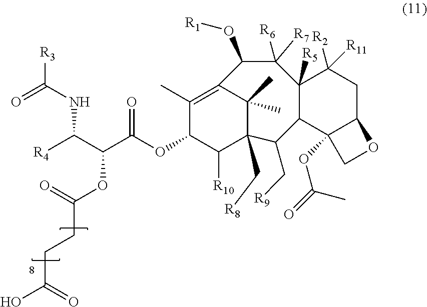

wherein:
R₁, R₃, R₄, R₅ and R₉, which are the same or different, are selected from H, alkyl, alkenyl, alkynyl, cycloalkyl, aryl and heteroaryl, the substituents of which can in turn be substituted by or alternated with one or more groups G which are the same as or different from one another, wherein G=—OH, —SH, —NH₂, halogen, —CN, —NO₂, —OR₁₂, —SR₁₂, —NHR₁₂, —NR12R13>C═O, >C═S, >C═NH, >C=NR₁₂, —C(O) R₁₂, —C(O) H—C(O) OH, —C(O) NH₂, —C(O) halogen, —C(O) OR₁₂, —C(O) SR₁₂, —C(O) NHR₁₂, —C(O) NR₁₂R13, —OC(O) OR₁₂, —OC(O) NHR₁₂, —NHC(O) NHR₁₂ or —NHC(S) NHR₁₂;
R₂, R₆, R₇, R₁₀ and R₁₁, which are the same or different, are selected from H, alkyl, alkenyl, alkynyl, cycloalkyl, aryl, heteroaryl, —OH, —SH, —NH₂, —NH— or —N<, the substituents of which can be in turn substituted by or alternated with one or more groups G′ which are the same as or different from one another, wherein G′=—OH, —SH, —NH₂, halogen, —CN, —NO₂, —OR₁₂, —SR₁₂, —NHR₁₂, —NR₁₂R₁₃, >C═O, >C═S, >C═NH, >C=NR₁₂, —C(O) R₁₂, —C(O) H—C(O) OH, —C(O) NH₂, —C(O) halogen, —C(O) OR₁₂, —C(O) SR₁₂, —C(O) NHR₁₂, —C(O) NR₁₂R₁₃, —OC(O) OR₁₂, —OC(O) NHR₁₂, —NHC(O) NHR₁₂ or —NHC(S) NHR₁₂;
R₅=H, alkyl, alkenyl, alkynyl or cycloalkyl;
R₁₂ and R₁₃, which are the same or different, are selected from H, alkyl, alkenyl, alkynyl, cycloalkyl, aryl and heteroaryl, which can in turn be substituted by or alternated with one or more groups X which are the same as or different from one another, wherein X=—CH₃, —CH₂—, —CH<, —OH, —SH, —NH₂, halogen, —CN, —NO₂, —O—, —S—, —NH—, —N<, >C=O, >C═S, >C═NH, >C═N—, —C(O) H—C(O) OH, —C(O) NH₂, —C(O) halogen, —C(O)O—, —C(O)S—, —C(O) NH—, —C(O) N<, —OC(O)O—, —OC(O) NH—, —NHC(O) NH— or —NHC(S) NH—;
R₂ and R₅ together can form a 3, 4, 5 or 6-membered cycloalkyl, optionally substituted by or alternated with one or more groups G, wherein G is as above defined;
R₆ and R⁷ together can form a group ═Y, wherein Y=CH₂, —O, ═S, ═NH or ═N—;
R1 and R₆ together can form a 3, 4, 5 or 6-membered cycloalkyl optionally substituted by or alternated with one or more groups G which are the same or different, wherein G is as above defined;
R₈ and R₁₀ together can form a 3, 4, 5 or 6-membered cycloalkyl, optionally substituted by or alternated with one or more groups G which are the same or different, wherein G is as above defined.

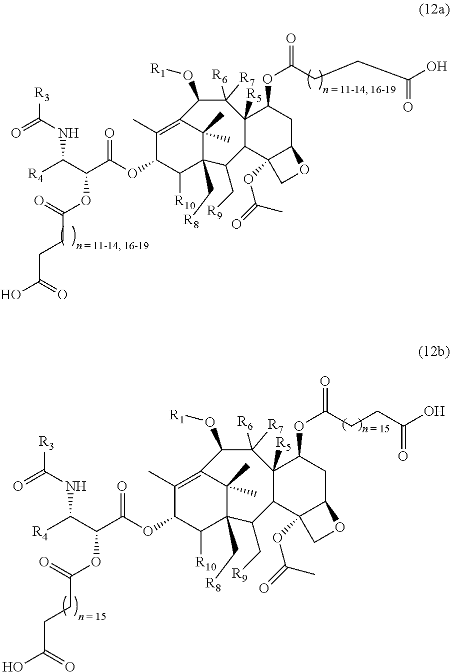

The term “alkyl” defines a straight or branched C₁-C₂₀ chain, the term “alkenyl” defines a straight or branched C₂-C₂₀ chain containing at least one carbon-carbon double bond, the term “alkynyl” defines a straight or branched C₂-C₂₀ chain containing at least one carbon-carbon triple bond, the term “aryl” defines cyclic or polycyclic C₃-C₂₀ aromatic systems, and the term “heteroaryl” defines cyclic or polycyclic C₃-C₂₀ aromatic systems containing at least one atom of N, O or S.

For clarity, the term “taxanes” defines compounds containing a skeleton of Formula (13)

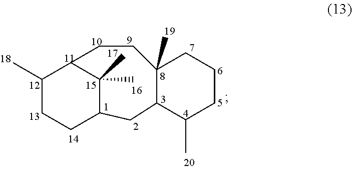

The process according to the invention provides the compounds of Formula (11) with high purity, where “high purity” means the presence of impurities of Formulas (12a) and (12b) in an amount less than that reported in the prior art; preferably an amount of impurities measured by HPLC analysis not exceeding 10%, 9%, 8%, 7%, 6%, 5%, 4%, 3%, 2%, 1%, 0.5% or 0.1%; even more preferably the absence of impurities of Formulas (12a) and (12b). Said compounds constitute a further aspect of the present invention. The formation of said impurities is attributable to the presence of compounds of Formula (6a) in the compound of Formula (6)

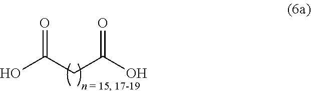

In particular, the process provides the compound of Formula (5) with a content of the impurity of Formula (8) not exceeding 10%, 9%, 8%, 7%, 6%, 5%, 4%, 3%, 2%, 1%, 0.5% or 0.1% measured by HPLC analysis; preferably, the compound of Formula (5) without the impurity of Formula (8). Said compound is a further aspect of the present invention.

In a first aspect thereof, the invention relates to a process for the preparation of a compound of Formula (11), which comprises
a) reacting a compound of Formula (14)

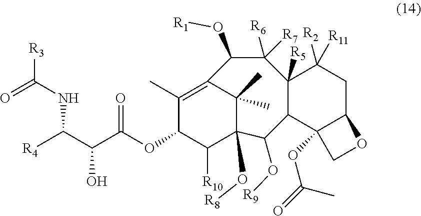

wherein R₁-R₁₃ are defined as above;
with a compound of Formula (15)

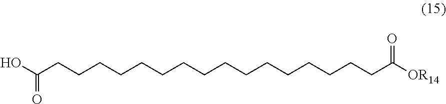

wherein R₁₄ is a protecting group removable by catalysis with a transition metal and
b) removing the protecting group.
Compounds of Formula (14) are preferably used

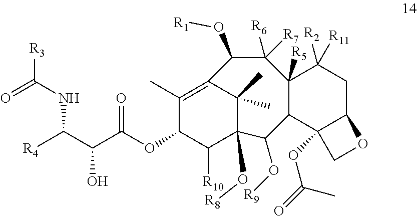

wherein:
R₁. R₃, R₄, R₈ and R₉, which are the same or different, are selected from H, C₁-C₁₀ alkyl, C₂-C₁₀ alkenyl, C₂-C₁₀ alkynyl, C₃-C₈ cycloalkyl, C₆-C₁₀ aryl and C₃-C₁₀ heteroaryl, which are optionally substituted by or alternated with one or more groups G which are the same or different, wherein G is as above defined;
R₂, R₆, R₇, R₁₀ and Ru, which are the same or different, are selected from H, C₁-C₁₀ alkyl, C₂-C₁₀ alkenyl, C₂-C₁₀ alkynyl, C₃-C₈ cycloalkyl, C₃-C₁₀ aryl, C₃-C₁₀ heteroaryl, —OH, —SH, —NH₂, —NH—, and —N<, which are optionally substituted by or alternated with one or more groups G′ which are the same or different, wherein G′ is as above defined;
R₅=H, C₁-C₅ alkyl, C₂-C₅ alkenyl, C₂-C₅ alkynyl or C₃-C₆ cycloalkyl;
R₂ and R₅ together can form a 3, 4, 5 or 6-membered cycloalkyl, optionally substituted by or alternated with one or more groups G, wherein G is as above defined;
R₆ and R⁷ together can form a group ═Y, wherein Y=CH₂, ═O, ═S, ═NH or ═N—;
R₁ and R₆ together can form a 3, 4, 5 or 6-membered cycloalkyl, optionally substituted by or alternated with one or more groups G which are the same or different, wherein G is as above defined.

More preferably, the compounds of Formula (14) are selected from paclitaxel (2), docetaxel (3), cabazitaxel (4), larotaxel (16), ortataxel (17), BMS-184476(18), tesetaxel (19), milataxel (20), SB-T-1214(21), SB-T-1216(22), SB-T-121602(23), SB-T-12854 (24) and docetaxel-f3-t-Boc (25); more preferably, they are selected from paclitaxel (2), docetaxel (3), ortataxel (17), tesetaxel (19), SB-T-1214(21), SB-T-1216(22), SB-T-121602(23), SB-T-12854(24) and docetaxel-f3-t-Boc (25); even more preferably, they are selected from paclitaxel (2) and docetaxel (3)

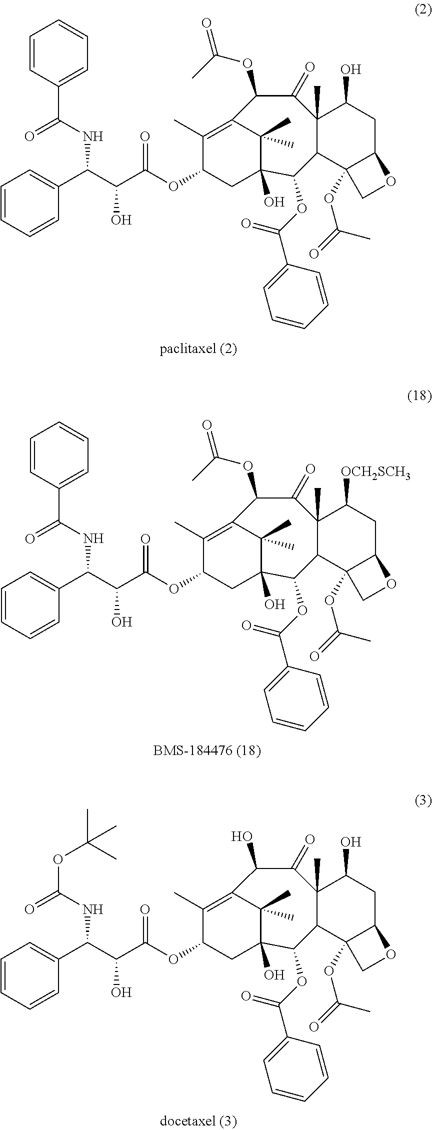

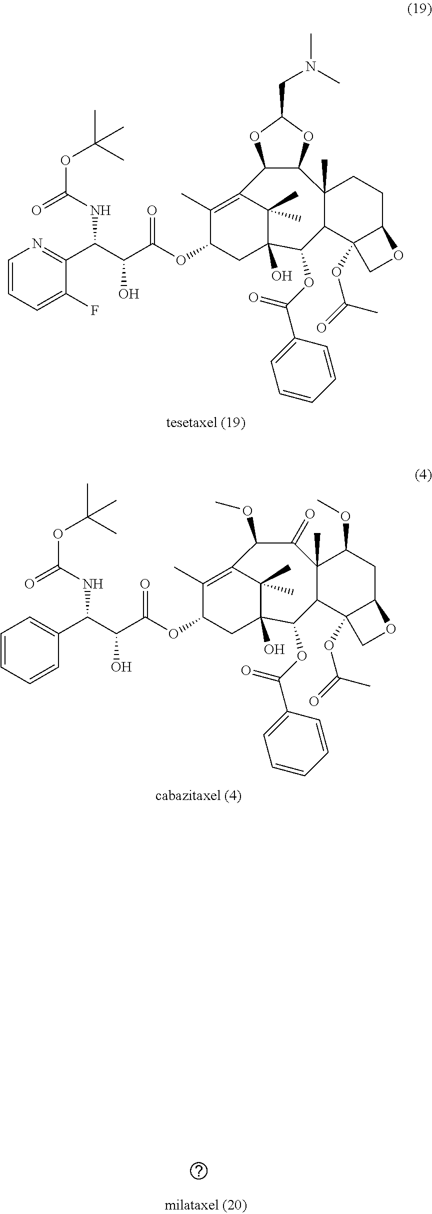

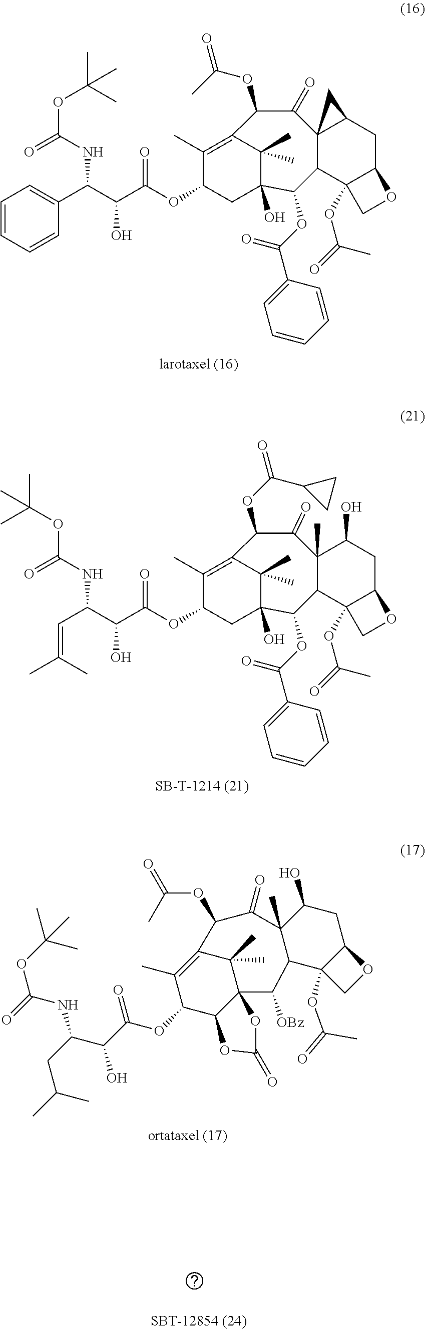

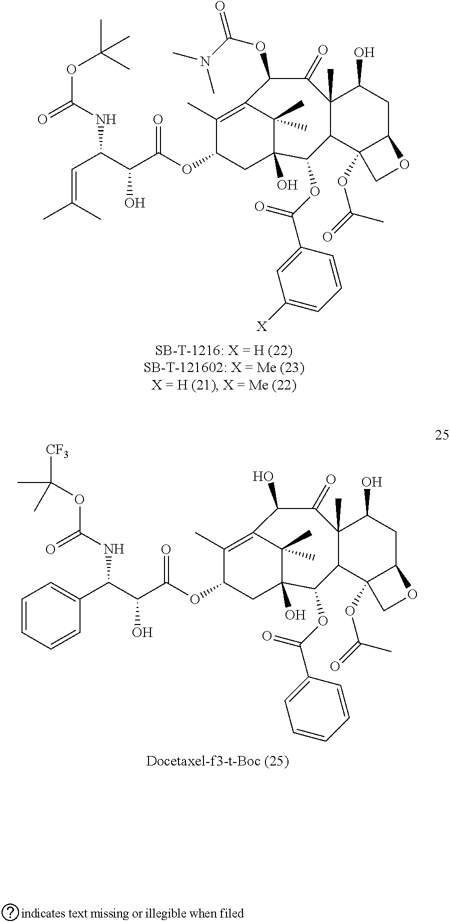

Compounds of Formula (15) are preferably used

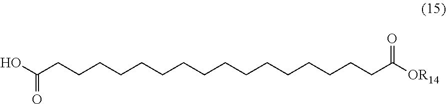

wherein R₁₄ is allyl, or 1,1-dimethyl-2-butenyl; more preferably a compound of Formula (15) is used

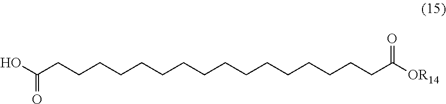

wherein R₁₄ is allyl.

The compounds of Formula (15), in particular the compound wherein R₁₄ is allyl, represent a second aspect of the invention.

Typically, the compound of Formula (15) wherein R₁₄=allyl is characterised as follows:
the XRPD spectrum exhibits a crystalline structure and comprises distinctive reflections, expressed as 20° angles, with a relative intensity greater than or equal to 5%, approximately amounting to 4.95; 8.27; 21.33(+0.2). In detail, the XRPD spectrum of the compound of Formula (15) exhibits a crystalline structure and comprises distinctive reflections, expressed as 20° angles, with a relative intensity greater than 1%, approximately amounting to 3.22, 4.95, 6.60, 8.27, 11.61, 13.29, 20.02, 20.75, 21.33, 21.97, 23.41, 24.23, 37.21, 38.98, 40.75 (+0.2). In even more detail, the XRPD spectrum of the compound of Formula (15) exhibits a crystalline structure and comprises distinctive reflections, expressed as 20° angles, approximately amounting to 3.22, 4.95, 6.60, 8.27, 9.93, 11.61, 13.29, 20.02, 20.75, 21.33, 21.97, 23.41, 24.23, 37.21, 30.20, 33.73, 34.20, 38.98, 40.75, 42.57(+0.2);
the NMR spectrum exhibits peaks at 5.9, 5.2, 4.5, 2.3-2.1, 1.5, 1.2 ppm.

In a third aspect thereof, the invention relates to a process for the preparation of a compound of Formula (15).

With special reference to the first aspect, the invention relates to a process comprising the following steps:
a) reacting the compound of Formula (15) with a compound of Formula (14) in the presence of a condensing agent to give compound (26)

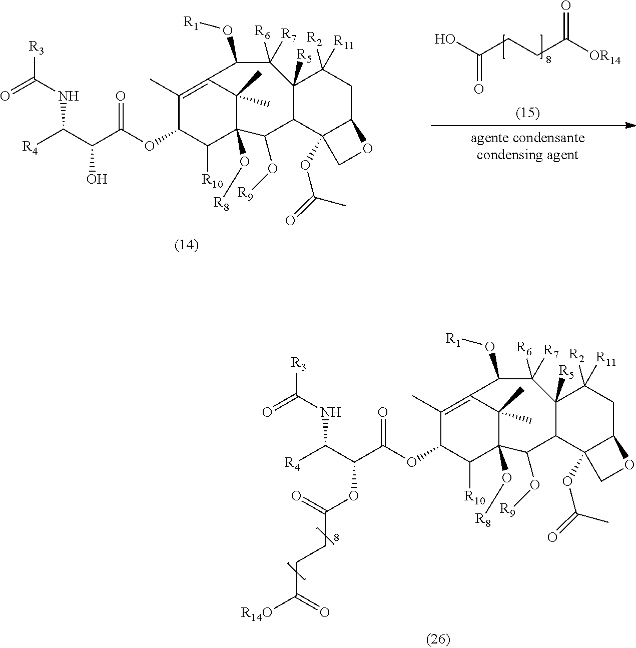

b) removing group R₁₄ from compound (26) by catalysis with a transition metal to give a compound of Formula (11)

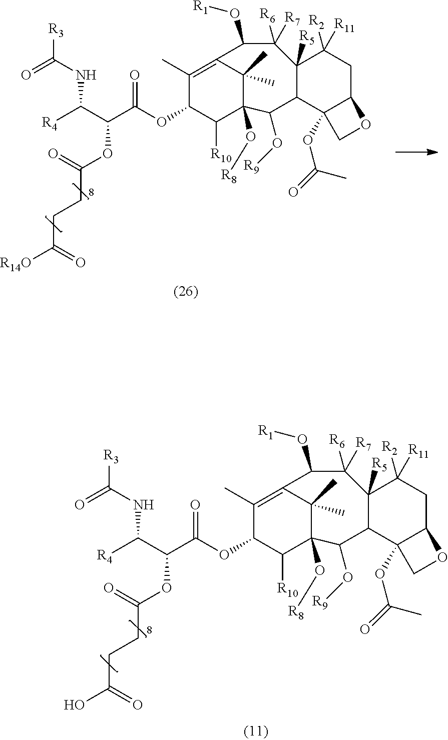

In a more preferred aspect, the invention relates to a process for the preparation of ODDA-paclitaxel (5) comprising the following steps:
a′) reacting a compound of Formula (15) with paclitaxel (2) in the presence of a condensing agent to give compound (27);

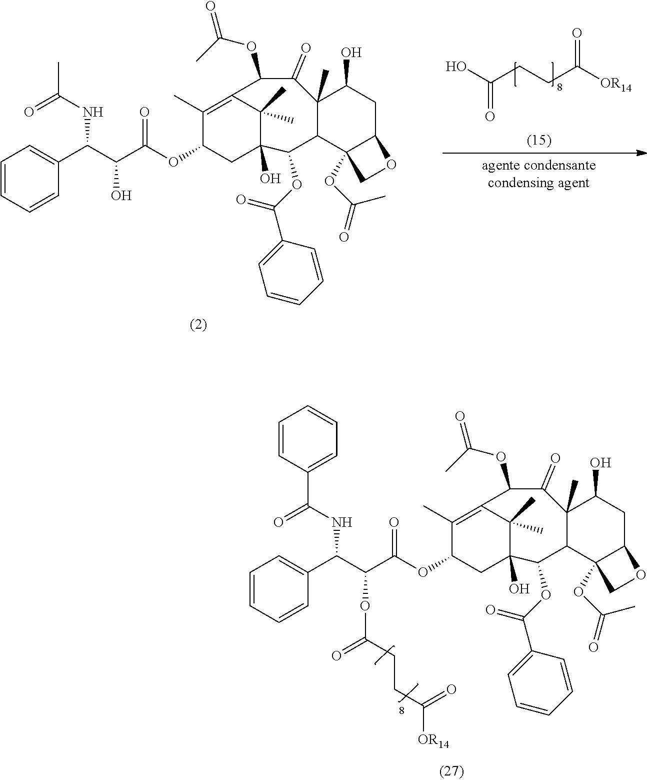

b′) removing group R₁₄ by catalysis with a transition metal to give an ODDA-paclitaxel of Formula (5)

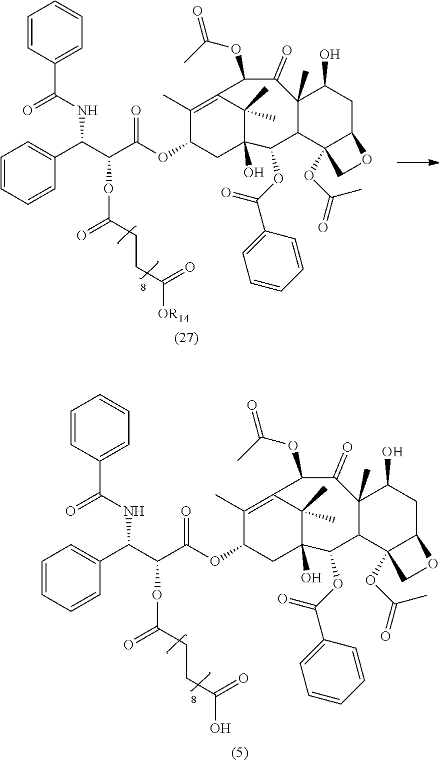

In steps a) and a′), the condensing agent used is typically selected from N,N′-dicyclohexylcarbodiimide (DCC), ethyl dimethylaminopropyl carbodiimide (EDC), N,N′-diisopropylcarbodiimide (DIC), N,N′-di-tert-butylcarbodiimide, 1,3-bis(2,2-dimethyl-1,3-dioxolan-4-ylmethyl) carbodiimide (BDDC) and N-cyclohexyl-N′-(2-morpholinoethyl)-carbodiimide methyl-p-toluenesulphonate (CMC); preferably ethyl dimethylaminopropyl carbodiimide (EDC).

In steps a) and a′), the solvent used is typically selected from dichloromethane (CH₂Cl₂), tetrahydrofuran (THF), dimethylformamide (DMF), dimethylacetamide (DMA) and dimethylsulphoxide (DMSO); preferably dichloromethane (CH₂Cl₂).

In steps a) and a′), the reaction mixture is maintained at a temperature ranging between 0° C. and 50° C.; preferably between 20° C. and 30.

In steps b) and b′), a transition metal is used as catalyst to remove the protecting group, preferably a palladium catalyst selected from Pd(Ph₃)₄. Pd(dba)₂. Pd₂(dba)₃. PdCl₂, Pd(OAc)₂ e PdCl₂(PPh₃)₂.

In steps b) and b′), the metal catalyst is used in combination with an organic base such as morpholine, pyridine or pyrrolidine or in combination with triphenylphosphine (PPh₃) and a reducing agent such as ammonium formate or triethylammonium formate.

Typically, Pd(pH₃)₄ is used in combination with an organic base such as morpholine, pyridine or pyrrolidine; Pd(dba)₂ and Pd₂(dba)₃ are used in combination with 2-3 equivalents of PPh₃ and with an organic base such as morpholine, pyridine or pyrrolidine; PdCl₂, Pd(OAc)₂ and PdCl₂(PPh₃)₂ are used in combination with 2-3 equivalents of PPh₃ and a reducing agent such as ammonium formate or triethylammonium formate.

Said mild deprotection conditions are particularly advantageous because, unlike the known conditions, they avoid the use of fluorinated reagents, which are highly toxic and require ad hoc industrial-scale facilities. Moreover, said metal catalysts enable the protecting group to be removed in a highly selective manner, without affecting the other ester functions present in the compounds of Formulas (26) and (27), and therefore also preventing the formation of further undesirable impurities.

The catalyst used is removed at the end of the reaction by washing with an aqueous solution of cysteine, cysteine hydrochloride, N-acetylcysteine or charcoal.

The compounds of Formula (14) obtained in step b), and ODDA-paclitaxel (5) obtained in step b′), are isolated after dissolution in an alcohol such as methanol, ethanol or isopropanol, preferably methanol, and precipitation, preferably by precipitation from an anti-solvent such as water. This step is particularly advantageous because in the known processes, ODDA-PTX (5) is obtained after concentration until dry by removing the solvent, in the form of a vitreous solid, whereas in the process according to the present invention it is obtained, after precipitation and filtration, in powder form. The isolation of compound (5) by precipitation and filtration greatly simplifies the process, because it reduces the times and costs involved in concentration until dry, and also provides the product in a non-vitreous form; solids obtained in said form are usually difficult to manage on an industrial scale because, in view of their extreme hardness, they can damage equipment and are difficult to recover, leading to loss of product or the need for repeated dissolution and concentration steps.

Between steps a)-b) and steps a′)-b′), the process according to the invention involves dissolving the crude reaction product obtained in steps a)-a′) in a water-alcohol solution, obtained by mixing water and an alcohol selected from methanol, ethanol, propanol, isopropanol, butanol, isobutanol and tert-butanol, preferably methanol, and washing the resulting solution with an apolar solvent selected from pentane, hexane, heptane, cyclohexane and toluene, preferably heptane.

When the starting compound of Formula (14) is paclitaxel (2), washing with an apolar solvent is surprisingly advantageous, because it enables the compound ODDA-PTX-ODDA of Formula (8) to be selectively eliminated without any loss of the compound of Formula (27); in more detail, the ODDA-PTX of Formula (5) obtained by the process according to the present invention is characterised by a much lower impurity content than an ODDA-PTX of Formula (5) obtained by a known process, such as the process described in WO2021/007322; as will be seen from the comparison set out in Table 1:

| | Process according to | Process according to |
|---|---|---|
| Compound | WO 2021/007322 | the invention |
| HOOC—(CH₂)₁₆-PTX | 0.19% | — |
| ODDA-PTX (5) | 47.8% | 95.0-97.0% |
| HOOC—(CH₂)₁₈-PTX | 0.39% | 0.04% |
| Mixture of ODDA-PTX | 15.8% | 0.6% |
| isomers and epimers | | |
| HOOC—(CH₂)₁₉-PTX | 1.06% | — |
| HOOC—(CH₂)₂₀-PTX | 0.09% | — |
| ODDA-PTX-ODDA (8) | 21.4% | 0.1-0.7% |

For clarity, “mixture of ODDAPTX isomers and epimers” means a mixture of compounds with a molecular weight equal to that of ODDA-PTX, analysed by LC_MS, but not individually separated and identified.

By means of the process according to the present invention, ODDA-PTX of Formula (5) is obtained with a yield of 98% and a purity of 95-97% measured by HPLC analysis according to the method reported in the experimental section. Said process also enables ODDA-paclitaxel (5) to be obtained in amorphous form.

With special reference to the third aspect, compound (15) is obtained by reacting the compound of Formula (6) with an alcohol of Formula (28), wherein R₁₄ is preferably selected from allyl, benzyl and 1,1-dimethyl-2-butenyl, catalysed by a Lewis or Brønstedt acid, such as p-toluenesulphonic acid, sulphuric acid, HfCl₄, FeCl₃, Sc (OTf)₃ and thio (acac)₂, preferably p-toluenesulphonic acid, to give the compound of Formula (15)

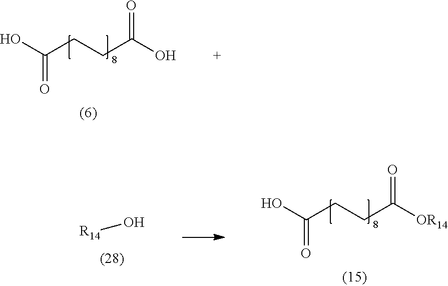

Typically, the alcohol of Formula (28) can be used in an amount ranging between 0.5 and 5 equivalents, preferably 2 equivalents.

Typically, the solvent used is selected from xylene, mesitylene and ethylbenzene; preferably toluene.

Typically, the reaction mixture is heated to a temperature ranging between 5° and 150° C.; preferably between 8° and 110° C.

Typically, the reaction mixture is cooled to a temperature ranging between-10 and 30° C.; preferably between 2° and 25° C.

The protection process of the compound of Formula (6) according to the invention is particularly advantageous; in fact, whereas the protection described in WO 2017/053391 and WO 2021/007322 requires a chromatography step for the purification and isolation of the resulting compound of Formula (9), the compounds of Formula (15) are purified by successive extractions with solvent and a final crystallisation. In particular, in a first embodiment, the process of purification and isolation of the compounds of Formula (15) comprises a step involving crystallisation of said compounds in a straight or branched C₂-C₅ alkyl alcohol; the process of purification and isolation of the compounds of Formula (15) preferably comprises the following steps:
l) dissolving a compound of Formula (15) in a straight or branched C₂-C₅ alkyl alcohol at a temperature ranging between 25 and 80° C. to give a solution B1;
m) crystallising the compound of Formula (15) by cooling solution B1 to give a suspension C₁;
n) isolating the crystallised compound of Formula (15) from suspension C₁.

Typically, in step 1), the alcohol is 2-propanol; said alcohol can be used pure or mixed with an organic base selected from Et₃N, pyridine, DMAP and imidazole; preferably Et₃N. If 2-propanol is mixed with a base, said base is present in the amount of 0.5-2.5 equivalents of the acid used to catalyse the reaction between the compound of Formula (6) and the compound of Formula (28); preferably in an amount ranging between 0.8 and 1.4 equivalents. If 2-propanol is used pure, step 1) is preceded by crystallisation of a crude compound of Formula (15) from a polar aprotic solvent selected from DMF, DMSO, acetone and ethyl acetate; preferably acetone.

Typically, in step 1), the solution is heated to a temperature ranging between 4° and 45° C.

Typically, in step m), the solution is cooled to room temperature; said temperature can be reached directly or in one or more steps involving maintaining the solution at a temperature ranging between room temperature and 45° C.; the solution is preferably cooled from 45° C. to 40° C., from 40° C. to 34° C., and from 34° C. to room temperature.

In another embodiment, the process of purification and isolation of the compounds of Formula (15), preferably of the compound wherein R₁₄ is allyl, comprises the following steps:
c) dissolving the crude compound of Formula (15) in a basic water-alcohol solution to give solution A;
d) washing solution A with an apolar solvent to give a solution B (water-alcohol phase) and a solution C (organic phase);
e) acidifying solution B to pH=3 to give a suspension D;
f) extracting suspension D with an apolar solvent to give a solution E;
g) concentrating solution E at low pressure to give a partly purified compound of Formula 15;
h) suspending the partly purified compound of Formula (15) in an alcohol to give a suspension F;
i) heating suspension F to give a solution G;
j) crystallising the compound of Formula (15) by cooling solution G to give a suspension H;
k) isolating the crystallised compound of Formula (15) from solution H.

For clarity, in step c), “basic water-alcohol solution” means a solution of water and an alcohol selected from methanol, ethanol, propanol and isopropanol, preferably methanol, to which an inorganic base selected from NaHCO₃ and KHCO₃ is added until a pH falling within the interval between 8 and 9 is reached.

Typically, in step d), the apolar solvent is selected from pentane, hexane, heptane, cyclohexane, toluene and benzene; preferably heptane.

Typically, in step e), the acid used is a mineral acid such as HF, HCl, HBr, H₂SO₄, HNO₃ or H₃PO₄; preferably HCl.

Typically, in step f), the apolar solvent is selected from pentane, hexane, heptane, cyclohexane, toluene and benzene; preferably, toluene.

Typically, in step h), the alcohol is selected from methanol, ethanol, propanol, isopropanol, butanol, iso-butanol and tert-butanol; preferably, methanol.

Typically, in step i), the solution is heated to between 3° and 90° C.; preferably between 35 and 60°; and more preferably to 40° C.

The protection processes of the compound of Formula (6) according to the invention are further advantageous because they allow a reduction in the amounts not only of the unreacted compound of Formula (6) and the compound of Formula (28), but also of the compounds of Formula (29), present as impurities in compound (6), the corresponding diprotected compounds of Formula (30), and the monoprotected compounds of Formula (31). In particular, the amount of the individual compounds (6) and (28)-(31), evaluated by HPLC analysis as Area %, is reduced by at least 20%, 25%, 30%, 35%, 40%, 45%, 50%, 55%, 60%, 65%, 70%, 75%, 80%, 85%, 90%, 95% or 99%. For clarity, the amount of the individual compounds is reduced independently of each other; for example, the amount of the compound of Formula (6) can be reduced by 90%, while the amount of the compound of Formula (30) can be reduced by 60%.

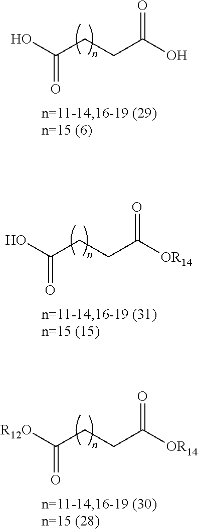

Moreover, elimination of the chromatography step, which is present in the known processes but does not allow the undesirable compounds (28)-(31) to be efficiently eliminated, is advantageous, because it gives rise to a more easily scalable process.

### Experimental Section

The following commercially available reagents and solvents were used:
absolute ethanol (“EtOH abs.”) (GC purity>99%);
ethanol (“EtOH”) (GC purity≥95%), (technical grade);
ethyl acetate (“AcOEt”) (GC purity>95%), (technical grade);
dichloromethane (“CH₂Cl₂”) (stabilised with amylene, purity≥98%);
potassium bicarbonate (“K₂CO₃”) (purity≥95%);
sodium chloride (“NaCl”) (purity≥95%), (technical grade);
37% hydrochloric acid (“HCl 37%);
toluene (“PhMe”) (HPLC purity≥99.7%);
methanol (“MeOH”) (HPLC purity≥99.9%);
tetrahydrofuran (“THF”) (HPLC purity≥99.9%);
palladium acetate (“Pd(OAc)₂”) 98%;
triphenylphosphine (“PPh₃”) (HPLC purity≥99%);
triethylamine (“Et₃N”) (GC purity≥99%);
formic acid (“HCOOH”) (purity≥98%);
allyl alcohol (GC purity≥99.5%);
heptane (GC purity≥99%).

The HPLC analyses were conducted with an HPLC apparatus consisting of a quaternary pump, a thermostated autosampler, a column compartment and a UV/VIS detector, using a Zorbax SB-C8 column (1=150 mm, i.d.=4.6 mm, particle size=3.5 μm) and solvents A (water+100 ppm HCOOH) and B (CH₃CN+100 ppm HCOOH) as mobile phase.

### Example 1-Preparation method of mono-allyl-octadecanedioic acid (allyl-ODDA)

Allyl alcohol (1.85 g, 31.8 mmol) was added to a solution of ODDA (5 g, 15.9 mmol) and pTSA·H₂O (150 mg, 0.79 mmol) in toluene (185 mL), heated to 90° C.; said solution was first maintained at reflux for 4 hours, thus removing the water by azeotropic distillation, then cooled to room temperature, filtered through celite, and finally, concentrated at low pressure. The resulting residue was dissolved in a MeOH/H₂O (150 mL) mixture at pH=8(1.26 g of KHCO₃) and washed with heptane (3×150 mL). The water-methanol phases were acidified to pH-3 by adding 1M HCl until a suspension was obtained, subsequently extracted with toluene (2×250 mL). The toluene phases were separated and concentrated at low pressure. The resulting residue was suspended in methanol, heated at 40° C. until completely dissolved, and finally, cooled to room temperature, to obtain a solid precipitate. Said solid, isolated as a white powder, was isolated by filtration and dried at 40° C. for 24 hours; (2.0 g, yield: 35%).

1H-NMR: 5.9 ppm (1H, m); 5.2 ppm (2H, m); 4.5 ppm (2H, m); 2.3-2.1 ppm (4H, m); 1.5 ppm (4H, m); 1.2 ppm (24H, m).

### Example 2-Preparation method of allyl-ODDA-paclitaxel

Paclitaxel (3.33 g, 3.90 mmol), mono-allyl-ODDA (1.38 g, 3.90 mmol) and DMAP (52 mg, 0.43 mmol) were dissolved in CH₂Cl₂(27 mL) in a nitrogen atmosphere; a solution of EDC.HCl (1.34 g, 6.98 mmol) in CH₂Cl₂(27 mL) was added to said solution, drop by drop, in 30 minutes. The resulting reaction, after being maintained under stirring at room temperature for 1.5 hours, was washed with 0.5M HCl (2×50 mL) and 15% saturated solution of NaCl (50 mL); the organic phases were combined and concentrated at low pressure The resulting solid was dissolved in MeOH (60 mL), and water (6 mL) was dropped into said solution; the water-alcohol solution was washed with heptane (3×30 mL), then with saturated solution of NaCl (70 mL), and finally, extracted with CH₂Cl₂(3×50 mL). After low-pressure concentration and drying of the combined organic phases, a vitreous white solid was obtained (4.43 g, yield=95%).

### Example 3-Preparation method of ODDA-paclitaxel

In a nitrogen atmosphere, a mixture of HCOOH (0.28 mL, 7.44 mmol) and Et₃N (1.28 mL, 9.21 mmol) in THF (3.6 mL) was added at room temperature to a solution of Pd(OAc)₂(8.3 mg, 0.04 mmol) and PPh₃(19.5 mg, 0.07 mmol) in THF (1.5 mL); a diallyl-ODDA-paclitaxel solution was added to said mixture, maintained under vigorous stirring, and the resulting mixture was stirred until complete conversion. The reaction mixture was then diluted with AcOEt (50 mL), and washed with 0.5M HCl (50 mL) and saturated solution of NaCl (50 mL). The combined organic phases were concentrated under vacuum; the resulting residue was dissolved in CH₂Cl₂(20 mL) and washed with an aqueous solution of cysteine hydrochloride (0.3 g in 20 mL of water) at 40° C. for 24 hours. The organic phases were concentrated under vacuum; the resulting residue (4.1 g) was dissolved in EtOH (12 mL), and said solution was slowly dropped into water (36 mL), under stirring for one hour at room temperature, to obtain a suspension. Said suspension was then filtered and washed with a mixture of EtOH: H₂O (1:3, 8 mL). The solid was obtained as a white powder and dried under vacuum at 45° C. for 24 hours (2.82 g, yield=66%).

### Example 4-Preparation method of mono-allyl-octadecanedioic acid (allyl-ODDA)

ODDA (30 g, 95.4 mmol) and para-toluenesulphonic acid (0.9 g, 4.8 mmol) were suspended in toluene (1.1 L), and the resulting suspension was heated at 85-95° C. until a solution was obtained. Allyl alcohol (11 g, 189.4 mmol) was added to the solution in two aliquots, and the mixture was heated at reflux for 4 hours, removing the water by azeotropic distillation. The solution was left to cool to room temperature and filtered through celite. The filtered solution was then concentrated by vacuum distillation (to about 150 mL). Said concentrate was suspended in n-heptane (about 900 mL) and maintained under stirring at room temperature for about an hour; the suspension was then filtered, and the solid residue was washed twice with heptane (60 mL). After 18 hours in a stove at 50° C. under vacuum, 10.1g of allyl-ODDA was obtained.

### Example 5-Mono-allyl-octadecanedioic acid (allyl-ODDA) crystallisation method

Allyl-ODDA (10.0 g, 28.2 mmol) was suspended in a mixture of 2-propanol (60 mL) and 0.45M Et₃N (1 mL, 0.45 mmol); the resulting mixture was heated to 45° C. until a clear solution was obtained, then cooled first to 40° C. in two hours, then to 34° C. in 6 hours, and finally to room temperature overnight. The solid formed was filtered and washed with 2-propanol (10 mL). After 16 hours in a stove at 50° C. under vacuum, 8.1 g of allyl-ODDA was obtained (crystallisation yield=80%, yield vs starting ODDA=24%).

### Example 6-Preparation method of allyl-ODDA-paclitaxel

Paclitaxel (19.5 g, 22.8 mmol), mono-allyl-ODDA (8.1 g, 22.8 mmol) and DMAP (280 mg, 2.29 mmol) were suspended in CH₂Cl₂(157 mL) in a nitrogen atmosphere; a solution of EDC. HCl (7.9 g, 41.2 mmol) in CH₂Cl₂(157 mL) was added to said solution, drop by drop, in 30 minutes. The resulting reaction, after being maintained under stirring at room temperature for 24 hours, was washed with 0.5M HCl (310 mL) and 15% saturated solution of NaCl (310 mL); the organic phase was concentrated at low pressure. The resulting solid was dissolved in MeOH (390 mL), and water (40 mL) was dropped into said solution until a suspension was obtained; the water-alcohol suspension was washed with heptane (3×196 mL), then with 6% saturated solution of NaCl (390 mL), and finally, extracted with CH₂Cl₂(3×50 mL). After low-pressure concentration of the combined organic phases and drying, a spongy white solid was obtained (26 g, yield=95%).

### Example 7-Preparation method of ODDA-paclitaxel

In a nitrogen atmosphere, a mixture of HCOOH (1.68 mL, 44.5 mmol) and Et₃N (7.42 mL, 53.4 mmol) in THF (21 mL) was added at room temperature to a solution of Pd(OAc)₂(50 mg, 0.22 mmol) and PPh₃(115 mg, 0.44 mmol) in THF (4 mL); a solution of allyl-ODDA-paclitaxel (26 g, 21.8 mol) in THF (52 mL) was added to said mixture, maintained under vigorous stirring, and the resulting mixture was stirred, heating to 50° C., until complete conversion. The reaction mixture was then diluted with AcOEt (500 mL), and washed with 1M HCl (500 mL) and 6% saturated solution of NaCl (500 mL). The combined organic phases were concentrated under vacuum; the resulting residue was dissolved in CH₂Cl₂(50 mL) and washed with an aqueous solution of cysteine hydrochloride (5 g in 50 mL of water) at 40° C. for 24 hours. The organic phases were concentrated under vacuum until a solid was obtained; after 16 hours in a stove under vacuum at 50° C., 25 g of ODDA-PTX was obtained (yield=98%).

### Example 8(comparative)-Preparation method of mono-TIPS-octadecanedioic acid (TIPS-ODDA)

ODDA (5 g, 15.9 mmol) was dissolved in DMF (75 mL) at 60° C.; TIPS-CI (3.0 g, 15.6 mmol) and Et₃N (2.2 ml, 15.6 mmol) were added to said solution, and the resulting mixture was kept under stirring overnight. The mixture was filtered and dried. The residue was then purified on silica using a 1:50 THF: DMC mixture as eluent. 217 mg of TIPS-ODDA in the form of oil was obtained from the purification.

The remaining fractions obtained from the column were again purified by silica-gel column chromatography, using an elution gradient of THF: DMC 1:100→2.5:100. 1.28 g of TIPS-ODDA in the form of oil was obtained from the purification.

1. A process for preparing a compound of Formula (11) 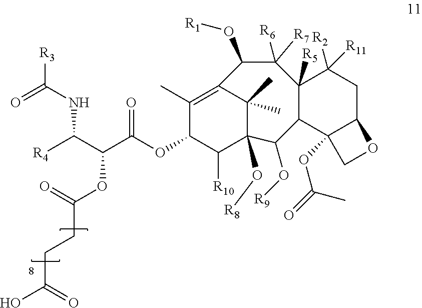
comprising:
a) reacting a taxane of Formula (14) 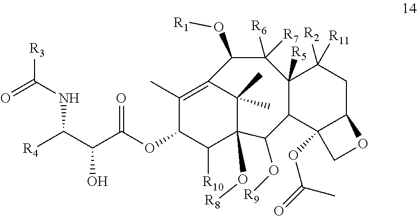
with a compound of Formula (15) 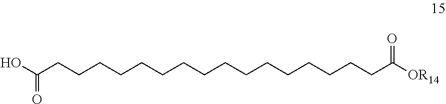
wherein
R₁. R₃, R₄, R₅ and R₉, which are the same or different, are selected from H, alkyl, alkenyl, alkynyl, cycloalkyl, aryl and heteroaryl, which may in turn be substituted by or alternated with one or more groups G which are the same as or different from one another, wherein G=—OH, —SH, —NH₂, halogen, —CN, —NO₂, —OR₁₂, —SR₁₂, —NHR₁₂, —NR₁₂R₁₃ >C=0, >C═S, >C═NH, >C=NR₁₂, —C(O) R₁₂, —C(O) H—C(O) OH, —C(O) NH₂, —C(O) halogen, —C(O) OR₁₂, —C(O) SR₁₂, —C(O) NHR₁₂, —C(O) NR₁₂R₁₃, —OC(O) OR₁₂, —OC(O) NHR₁₂, —NHC(O) NHR₁₂ or —NHC(S) NHR₁₂;
R₂, R₆, R₇, R₁₀ and R₁₁, which are the same or different, are selected from H, alkyl, alkenyl, alkynyl, cycloalkyl, aryl, heteroaryl, —OH, —SH, —NH₂, —NH— and —N<, which may in turn be substituted by or alternated with one or more groups G′ which are the same as or different from one another, wherein G′=—OH, —SH, —NH₂, halogen, —CN, —NO₂, —OR₁₂, —SR₁₂, —NHR₁₂, —NR₁₂R₁₃, >C═O, >C═S, >C═NH, >C=NR₁₂, —C(O) R₁₂, —C(O) H—C(O) OH, —C(O) NH₂, —C(O) halogen, —C(O) OR₁₂, —C(O) SR₁₂, —C(O) NHR₁₂, —C(O) NR₁₂R₁₃, —OC(O) OR₁₂, —OC(O) NHR₁₂, —NHC(O) NHR₁₂ or —NHC(S) NHR₁₂;
R₅=H, alkyl, alkenyl or cycloalkyl;
R₁₂ and R₁₃, which are the same or different, are selected from H, alkyl, alkenyl, alkynyl, cycloalkyl, aryl and heteroaryl, which may in turn be substituted by or alternated with one or more groups X which are the same as or different from one another, wherein —CH₃, —CH₂—, —CH<, —OH, —SH, —NH₂, halogen, —CN, —NO₂, —O—, —S—, —NH—, —N<, >C═O, >C=S, >C=NH, >C═N—, —C(O) H—C(O) OH, —C(O) NH₂, —C(O) halogen, —C(O)O—, —C(O)S—, —C(O) NH—, —C(O) N<, —OC(O)O—, —OC(O) NH—, —NHC(O) NH—, —NHC(S) NH—;
R₂ and R₅ together may form a 3, 4, 5 or 6-membered cycloalkyl, optionally substituted by or alternated with one or more groups G, wherein G is as above defined;
R₆ and R₇ together may form a group ═Y, wherein Y=CH₂, —O, ═S, ═NH, ═N—;
R₁ and R₆ together may form a 3, 4, 5 or 6-membered cycloalkyl, optionally substituted by or alternated with one or more groups G which are the same or different, wherein G is as above defined;
R₈ and R₁₀ together may form a 3, 4, 5 or 6-membered cycloalkyl, optionally substituted by or alternated with one or more groups G which are the same or different, wherein G is as above defined; and
R₁₄ is a protecting group that can be removed by catalysis with a transition metal, b) removing the protecting group.

2. The process according to claim 1, wherein in the taxane of Formula (14) 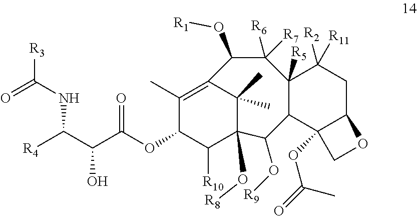
R₁, R₃, R₄, R₅ and R₉, which are the same or different, are selected from H, C₁-C₁₀ alkyl, C₂-C₁₀ alkenyl, C₂-C₁₀ alkynyl, C₃-C₈ cycloalkyl, C₆-C₁₀ aryl and C₃-C₁₀ heteroaryl, optionally substituted by or alternating with one or more groups G which are the same or different, wherein G is as above defined;
R₂, R₆, R₇, R₁₀ and R₁₁, which are the same or different, are selected from H, C₁-C₁₀ alkyl, C₂-C₁₀ alkenyl, C₂-C₁₀ alkynyl, C₃-C₈ cycloalkyl, C₆-C₁₀ aryl, C₃-C₁₀ heteroaryl, —OH, —SH, —NH₂, —NH— and —N<, optionally substituted by or alternated with one or more groups G′ which are the same or different, wherein G′ is as above defined;
R₅=H, C₁-C₈alkyl, C₂-C₅ alkenyl, C₂-C₅ alkynyl or C₃-C₆ cycloalkyl;
R₂ and R₅ together may form a 3, 4, 5 or 6-membered cycloalkyl, optionally substituted by or alternated with one or more groups G, wherein G is as above defined;
R₆ and R⁷ together may form a group ═Y, wherein Y=CH₂, O, S, NH or N—;
R₁ and R₆ together may form a 3, 4, 5 or 6-membered cycloalkyl, optionally substituted by or alternated with one or more groups G which are the same or different, wherein G is as above defined.

3. The process according to claim 1, wherein the taxane of Formula 14 is selected from paclitaxel (2), docetaxel (3), cabazitaxel (4), larotaxel (16), orthotaxel (17), BMS-184476(18), tesetaxel (19), milataxel (20), SB-T-1214(21), SB-T-1216(22), SB-T-121602(23), SB-T-12854(24) and docetaxel-f3-t-Boc (25)

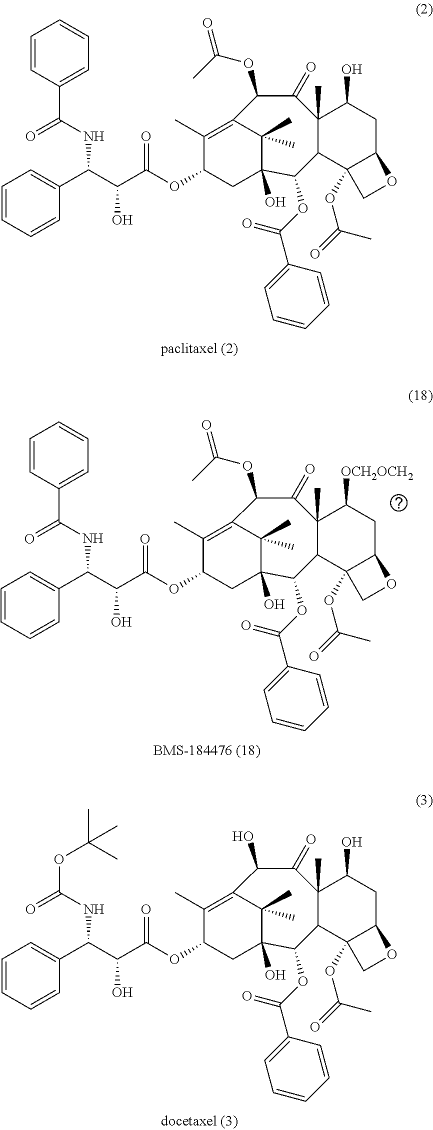

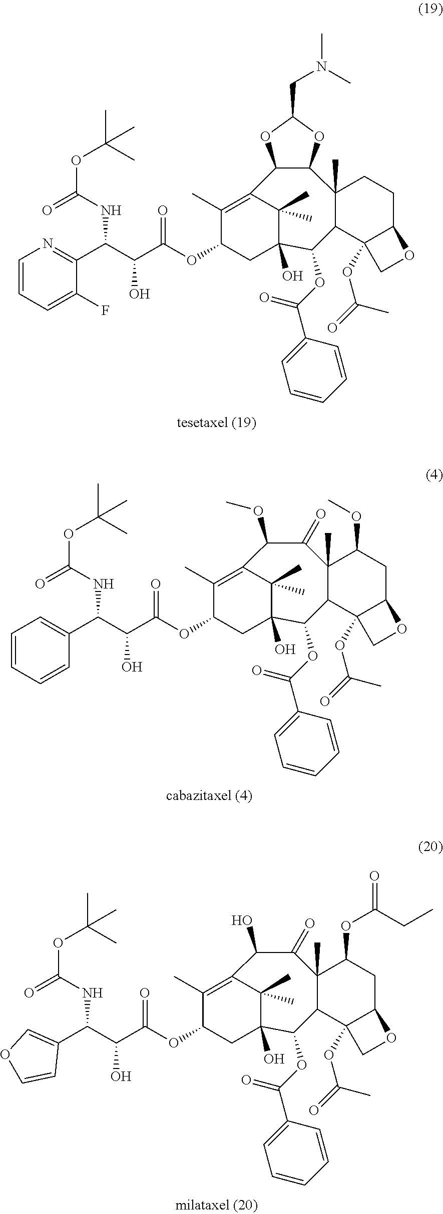

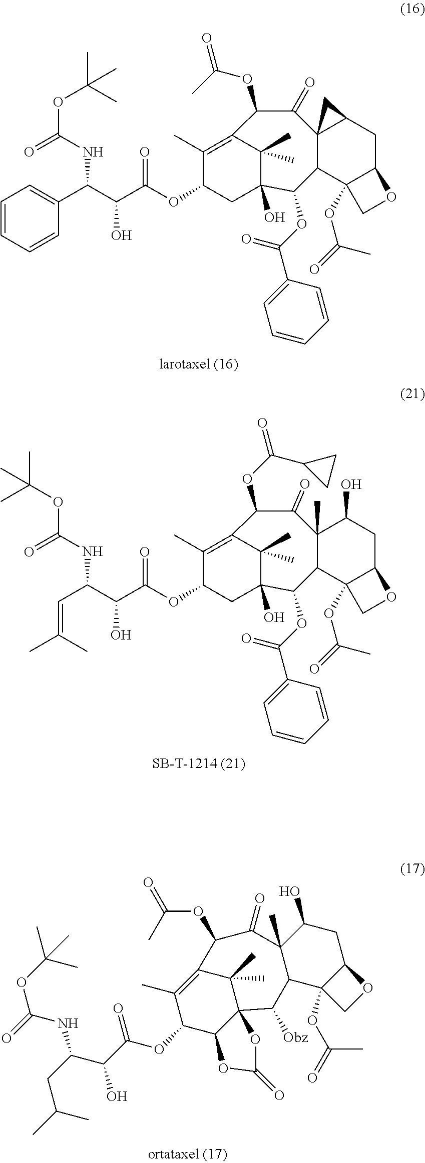

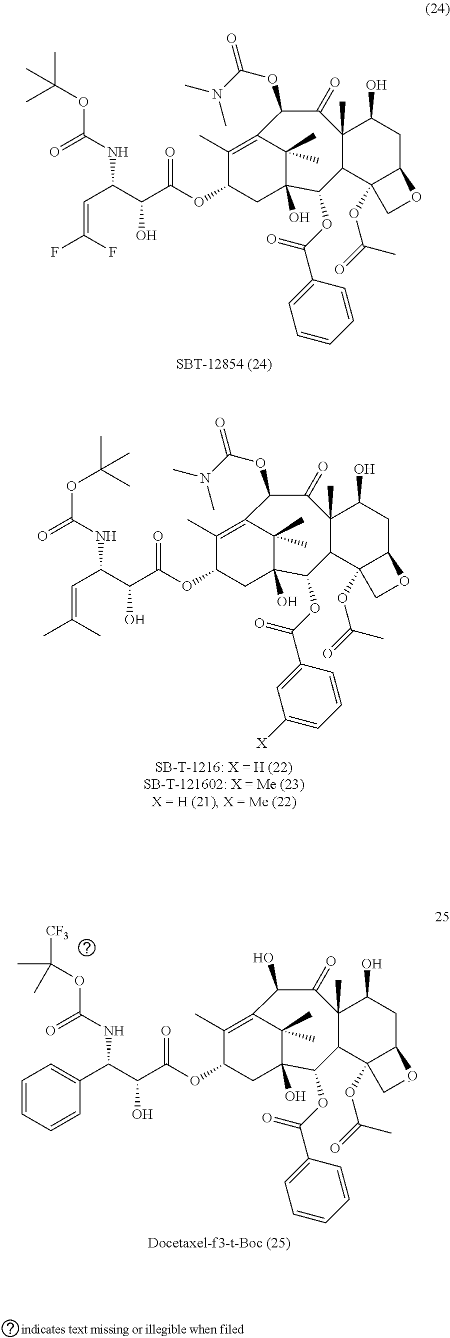

4. The process according to claim 1, wherein R₁₄ is allyl or 1,1-dimethyl-2-butenyl.

5. The process according to claim 1, wherein R₁₄ is allyl.

6. The process according to claim 1, comprising the following steps:
a) reacting a compound of Formula (15) with a compound of Formula (14) in the presence of a condensing agent to give compound (26) 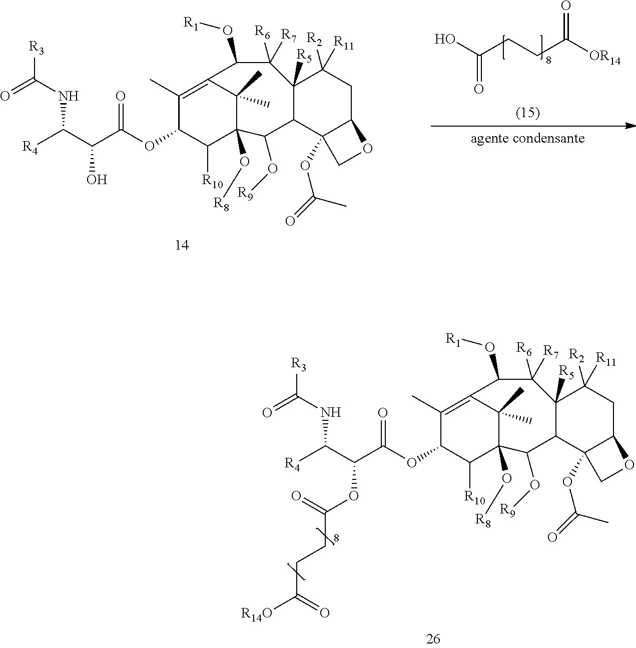
condensing agent;
b) removing group R₁₄ from compound (26) by catalysis with a transition metal to give ODDA-taxane of Formula (11)

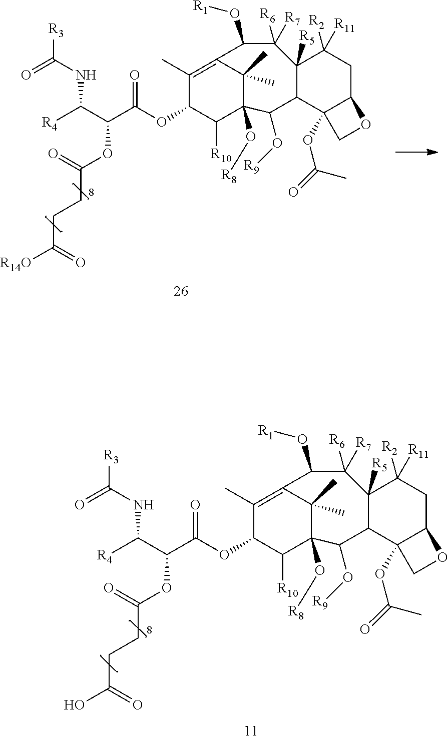

7. The process according to claim 6, comprising the following steps:
a′) reacting monoprotected octadecanedioic acid of Formula (15) with paclitaxel (2) in the presence of a condensing agent to give compound (27); 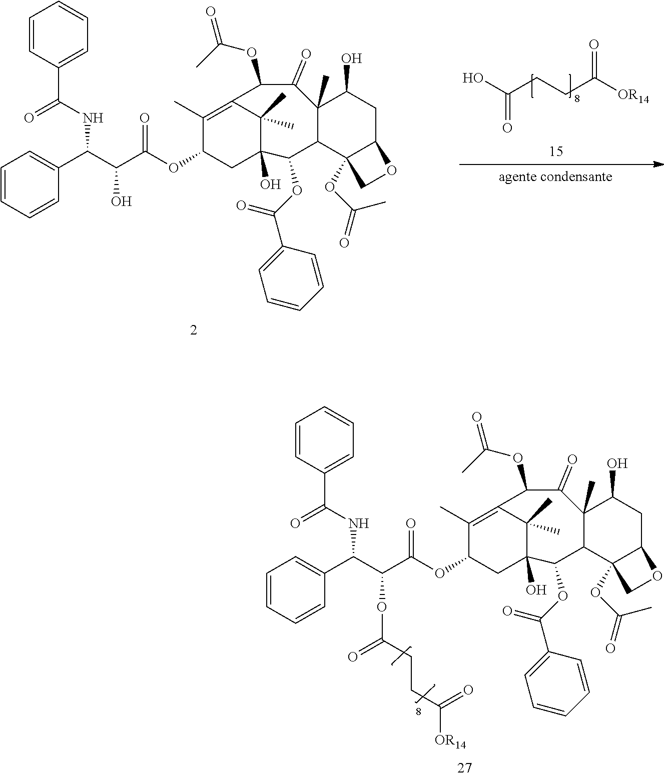
condensing agent;
b′) removing group R₁₄ by catalysis with a transition metal to give ODDA-paclitaxel of Formula (5)

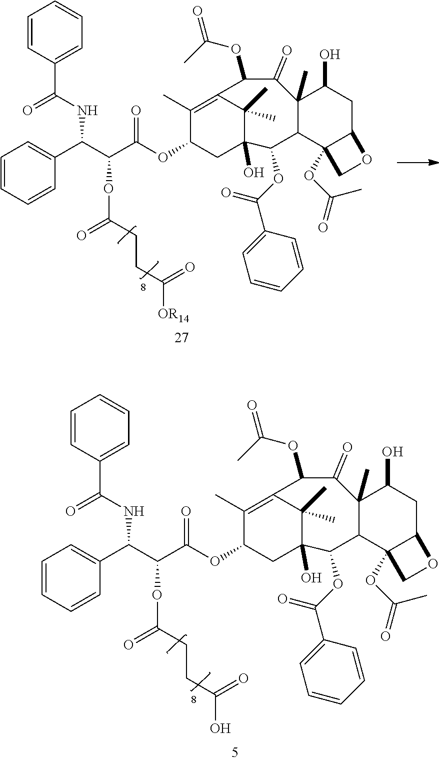

8. The process according to claim 1, wherein the transitional metal is selected from Pd(Ph₃) 4, Pd(dba)₂, Pd₂(dba)₃, PdCl₂, Pd(OAc)₂ and PdCl₂(PPh₃)₂.

9. The process according to claim 1, comprising washing of the compound of Formula (11) with an apolar solvent selected from pentane, hexane, heptane, cyclohexane and toluene.

10. The process according to claim 1, comprising isolating the compound of Formula (11) by precipitation.

11. The process according to claim 6, wherein the compounds of Formula (11) contain less than 10% of compounds of Formulas (12a) and (12b)

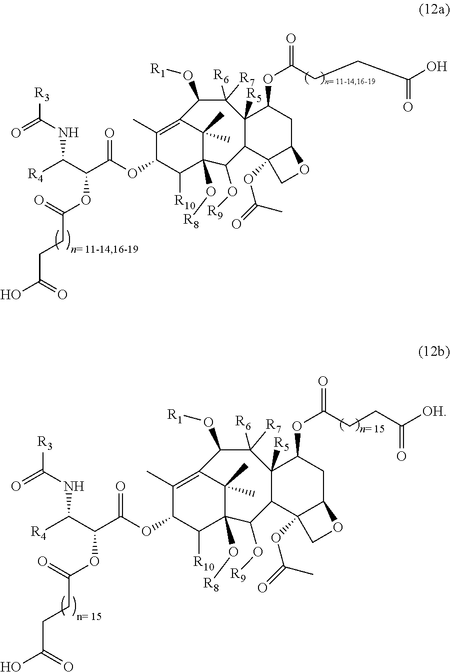

12. The process according to claim 7, wherein the compound of Formula (5) contains less than 10% of compounds of Formula (8)

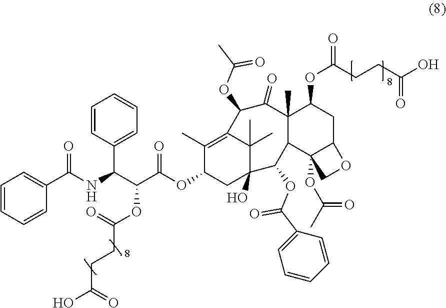

13. A process for preparing a compound of Formula (15) 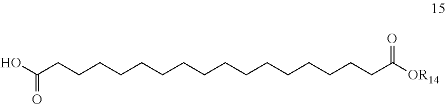
said process comprising reacting a compound of Formula (6) with a compound of Formula (28) 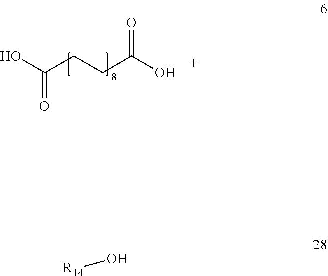
wherein R₁₄ is allyl, benzyl or 1,1-dimethyl-2-butenyl.

14. The process according to claim 13 comprising a crystallization step.

15. The process according to claim 14 comprising washing of the compound of Formula (15) with an apolar solvent selected from pentane, hexane, heptane, cyclohexane, toluene and benzene.

16. Compound of Formula (15) 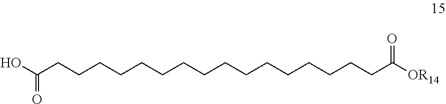
wherein R₁₄ is allyl or 1, 1-dimethyl-2-butenyl.

17. Amorphous form of ODDA-PTX (5).
